(19) United States
(12) Patent Application Publication
(10) Pub. No.: US 2013/0129838 A1
(43) Pub. Date: May 23, 2013

(54) NUTRITIONAL COMPOSITIONS AND METHODS FOR OPTIMIZING DIETARY ACID-BASE POTENTIAL

(71) Applicants: Kevin Burke Miller, Minneapolis, MN (US); Zamzam Kabiry Roughead, Plymouth, MN (US); Jennifer Rae Mager, St. Louis, MN (US); Douglas Richard Bolster, Eden Prairie, MN (US); Norman Alan Greenberg, New Hope, MN (US)

(72) Inventors: Kevin Burke Miller, Minneapolis, MN (US); Zamzam Kabiry Roughead, Plymouth, MN (US); Jennifer Rae Mager, St. Louis, MN (US); Douglas Richard Bolster, Eden Prairie, MN (US); Norman Alan Greenberg, New Hope, MN (US)

(73) Assignee: NESTEC S.A., Vevey (CH)

(21) Appl. No.: 13/518,296

(22) PCT Filed: Dec. 21, 2010
(86) PCT No.: PCT/US10/61444

§ 371 Date: Aug. 24, 2012

Related U.S. Application Data
(60) Provisional application No. 61/288,928, filed on Dec. 22, 2009.
(60) Provisional application No. 61/296,688, filed on Jan. 20, 2010.
(60) Provisional application No. 61/424,070, filed on Dec. 17, 2010.

(51) Int. Cl. A61K 33/06; G06F 17/10; A61K 33/42; A61K 38/16; A61K 33/08

(52) U.S. Cl. CPC A61K 33/06; A61K 38/168; A61K 33/08; A61K 33/42; G06F 17/10
USPC 424/601; 424/692; 424/682; 708/132

(57) ABSTRACT

Nutritional compositions having the potential to reduce metabolic acid load and methods of making and using the nutritional compositions are provided. In an embodiment, the present disclosure provides methods of selecting and administering nutritional compositions to patients. The methods may include modifications to calculating a metabolic acid potential of a nutritional composition, calculating a base content of a nutritional composition and subtracting the base content from the acid content to determine a potential renal acid load (“PRAL”) value. The present disclosure also provides computer implemented processes for predicting PRAL values.

### BACKGROUND

The present disclosure generally relates to health and nutrition. More specifically, the present disclosure relates to nutritional compositions having the potential to reduce metabolic acid load and methods of making and using the nutritional compositions to optimize and provide improved patient health, especially in individuals receiving long term tube feeding.

There are many types of nutritional compositions currently on the market. Nutritional compositions can be targeted toward certain consumer types, for example, young, elderly, athletes, and also those suffering from chronic or acute conditions or illnesses, etc., based on the specific ingredients of the nutritional composition. Nutritional compositions can also be formulated based on the certain physiological conditions that the nutritional compositions are intended to manage, treat or improve.

One goal of nutritional support is to improve metabolic disturbances in patients that may result from inactivity, a lack of variety in their diets or conditions which result in insufficiency in function of key organs. For example, patients who receive long-term tube-fed formulations often remain on a single dietary source for weeks, months or even years. As a result, the acid-base potential of the diet can play a significant role on the patient's health. Because tube fed patients are restricted in their dietary selections, and they may have renal insufficiency, the opportunity exists to positively influence acid-base balance through selective and targeted nutritional support. Patients may require specific nutritional compositions to prevent acid-base imbalance and for better management of their condition and/or to prevent onset of other chronic diseases (e.g., low bone mineral density, osteoporosis, skeletal muscle atrophy). Specific health benefits of improved acid-base balance through application of nutritional formulations include maintenance of bone, skeletal muscle and immune health, as well as improved pulmonary function. Improved acid-base balance through application of nutritional formulations may also include prevention of renal insufficiency and modulation of overt kidney disease. It is estimated that excess calciruia from excess diet acid load is 66 mg/day. If this calcium loss estimated from short term studies were extrapolated over time without adaptation a continuous loss of 66 mg/d would lead to 24 g per year or 480 g over 20 years. Adult humans have about 1150 g of calcium in their skeleton. See, Fenton et al., “Meta-analysis of the quantity of calcium excretion associated with the net acid excretion of the modern diet under the acid-ash diet hypothesis,” Am. J. Clin. Nutr., 88:1159-66 (2008). Therefore, calciuria associated with the modern' diet is sufficient in quantity that it could explain the progression of osteoporosis if the excess calcium is directly from bone.

Individuals expected to derive benefit from the application of the present disclosure include, for example, patients receiving long term tube feeding. Such individuals may include patients suffering from Alzheimer's, dementia, cognitive impairment and/or other neurodegenerative disorders including, for example, cerebral palsy, amyotrophic lateral sclerosis, and general neurological impairment. Individuals who are long term tube fed may experience formula-driven issues since many current tube feeding formulas lead to a range of complications including, for example, low grade acidosis.

Individuals expected to derive benefit from the application of the present disclosure may also include, for example, acutely ill individuals with renal compromise, elderly who are at risk of or experiencing musculoskeletal health problems, individuals in home care, bed-ridden persons, obese, obese with sleep apnea, individuals in a weight loss program trying to maintain lean body mass, pregnant women with elevated blood pressure, individuals with reduced respiration or respiratory capacity (including mechanically ventilated patients), individuals with metabolic or respiratory acidosis, diabetics including gestational diabetes, pediatrics with reduced renal and/or pulmonary function.

Examples of respiratory insufficiencies may include, for example, chronic obstructive pulmonary disease (“COPD”), chronic ventilation, congestive heart failure (“CHF”), emphysema, and respiratory failure caused by, for example, disease, trauma, brain damage, etc. Examples of renal insufficiencies include, but are not limited to, diabetes type 1 and 2, metabolic syndrome, aging, systemic lupus erythematosus, collagen diseases, renal damage, chronic dialysis, end stage renal disease, etc. Patients experiencing renal insufficiencies typically do not have a mineral-restricted diet, except for sodium, and a low acid ash diet such as diets discussed in the present disclosure, could prevent progression from renal insufficiency to chronic renal

In addition to the above-mentioned patient populations that may benefit from the application of the present disclosure, patients undergoing a full meal replacement therapy may also benefit. For example, total parenteral nutrition (“TPN”) is a way of supplying all the nutritional needs of the body by bypassing the digestive system and dripping nutrient solution directly into a vein. When a patient is fed via TPN, food is not supplied to the patient by any other routes. Enteral nutrition (“EN”) is a way to provide food through a tube placed in the nose, the stomach, or the small intestine. Some patients undergo a meal replacement therapy including both TPN and EN and may require diets such as those discussed in the present disclosure.

Patients subject to other sole liquid food diets may also benefit from the application of the present disclosure. Such patients include, for example, the elderly and individuals attempting to lose weight by consuming solely liquid products designed to restrict caloric intake, while providing the nutrients required by the body. An example of such a full meal replacement liquid product includes Nestlé S. A.'s OPTIFAST®.

Generally speaking, certain drugs administered via intravenous (“IV”) or oral routes can also lead to acidosis. As such, patient populations receiving such IV or oral drugs may also benefit from the application of the present disclosure.

One long term consequence of acid-base imbalance is thought to be development of osteoporosis through gradual loss of body calcium. Osteoporosis is a major public health threat characterized by low bone mass and fragility leading to increased risk of fractures. Osteoporosis affects an estimated 44 million Americans, or 55 percent of the people 50 years of age and older. To combat this debilitating disease, the public is advised to limit their protein, caffeine, phosphorus, and sodium intake based on the hypothesis that these factors adversely affect calcium metabolism. However, the basis of this advice, especially for protein and phosphorus, is controversial and the subject of much debate. Recent data show effects of protein and phosphorus to be opposite to what is predicted by the Remer and Manz calculations. See, Fenton et al., “Phosphate decreases urine calcium and increases calcium balance: a meta-analysis of the osteoporosis acid-ash diet hypothesis,” Nutrition J., 8:41 (2009). For every mole of phosphate, urine calcium decreases slightly by 0.004 mmol/d and calcium balanced is increased by 0.10 mmol/d. Epidemiologic studies examining the effects of protein on bone health have not been helpful in resolving the controversy and have also yielded mixed results.

### SUMMARY

Methods of formulations to decrease the acid load of nutritional composition are provided. Methods of making and using the nutritional compositions are also provided and include, for example, tube feed formulations, oral nutritional formulations, and modular formulations delivering low acid ash content.

The acid component is calculated using the equation (measured in mg/d for all, except as noted): acid content=[(P×0.0366)+(protein (g/day)×acid potential of the protein source(s) (mEq/100 g protein))+(Cl×0.0268)]. The base component of the nutritional composition is calculated using the equation: base content=[(Ca×0.0125)+(Mg×0.0263)+(K×0.0211)+(Na×0.0413)]. However, the observed effects of dietary P and Na are not consistent with the predicted values from the potential renal acid load (“PRAL”) formula. The present disclosure includes increases in the cations Ca, Mg, K plus P as the tools for increasing alkalinity of the diets.

Another method used to estimate net acid production is the protein to Potassium ratio. Increased values correlated with measures of renal net acid excretion (“RNAE”).

In an embodiment, the nutritional compositions include a source of protein. The protein source may be dietary protein including, but not limited to animal protein (such as milk protein, meat protein or egg protein), vegetable protein (such as soy protein, wheat protein, rice protein, canola and pea protein), or a combination thereof. In an embodiment, the protein is selected from the group consisting of pea, whey, chicken, corn, caseinate, wheat, flax, soy, carob, canola, pea or combinations thereof.

In an embodiment, the nutritional compositions include a source of carbohydrates. Any suitable carbohydrate may be used in the present nutritional compositions including, but not limited to, sucrose, lactose, glucose, fructose, corn syrup solids, maltodextrin, modified starch, amylose starch, tapioca starch, corn starch, isomalt, isomaltulose, or combinations thereof.

In an embodiment, the nutritional compositions include a source of fat. The source of fat may include any suitable fat or fat mixture. For example, the fat source may include, but is not limited to, vegetable fat (such as olive oil, corn oil, sunflower oil, rapeseed oil, hazelnut oil, soy oil, palm oil, coconut oil, canola oil, lecithins, and the like) and animal fats (such as milk fat), structured lipids or other modified fats such as medium chain triglycerides.

In an embodiment, the nutritional composition further includes one or more prebiotics and/or fiber (soluble and/or insoluble). As used herein, a “prebiotic” is preferably a food substance that selectively promotes the growth of beneficial bacteria or inhibits the growth or mucosal adhesion of pathogenic bacteria in the intestines. Prebiotics are not digested in the stomach and/or upper intestine or absorbed in the GI tract of the person ingesting them, but they are fermented by the gastrointestinal microflora and/or by probiotics. Prebiotics are for example defined by Glenn R. Gibson and Marcel B. Roberfroid, “Dietary Modulation of the Human Colonic Microbiota: Introducing the Concept of Prebiotics,” J. Nutr. 1995 125: 1401-1412. The prebiotic can be acacia gum, alpha glucan, arabinogalactans, arabinoxylans, beta glucan, dextrans, fructool igosaccharides, galactooligosaccharides, galactomannans, gentiooligosaccharides, glucooligosaccharides, guar gum, inulin, isonialtooligosaccharides, lactosucrose, lactulose, levan, maltodextrins, partially hydrolyzed guar gum, pecticoligosaccharides, resistant starches, retrograded starch, soy oligosaccharides, sugar alcohols, xylooligosaccharides, or their hydrolysates, or combinations thereof. Prebiotics are useful in the present compositions to enhance the uptake of cations (alkaline-ash minerals) as a result of short chain fatty acids produced during prebiotic fermentation.

In an embodiment, the nutritional composition further includes one or more probiotics. As used herein, probiotic micro-organisms (hereinafter “probiotics”) are preferably microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al “Probiotics: how should they be defined,” Trends Food Sci. Technol. 1999:10 107-10. In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. The probiotic can be of bacterial, yeast, or fungal origin, including Saccharomyces, Debaromyces, Candida, Pichia, Torulopsis, Aspergillus, Rhizopus, Mucor, Penicillium, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus or a combination thereof.

In another embodiment, the nutritional composition further includes one or more amino acids. The amino acid can be Isoleucine, Alanine, Leucine, Asparagine, Lysine, Aspartate, Methionine, Cysteine, Cystine, Phenylalanine, Glutamate, Threonine, Glutamine, Tryptophan, Citrulline, Glycine, Valine Proline, Serine Tyrosine, Arginine, Histidine or a combination thereof.

In an embodiment, the nutritional composition further includes one or more vitamin K₂ (menaquinone), synbiotics, fish oils, phytonutrients and/or antioxidants. The antioxidants can be, for example, vitamin A, carotenoids, vitamin C, vitamin E, selenium, flavonoids, Lactowolfberry, wolfberry, polyphenols, lycopene, lutein, lignan, coenzyme Q10 (“CoQ10”) and glutathione.

The nutritional composition includes minerals in a form that promotes metabolic alkalinity versus acidity; attached to various organic acids, amino or fatty acids, or naturally occurring as part of a real food. As an example, different forms of magnesium include: magnesium hydroxide (H₂MgO₂), magnesium phosphate tribasic (Mg₃(PO₄)₂), magnesium oxide (MgO), magnesium oleate (C₃₆H₆₆MgO₄).

The nutritional composition may further include free coenzyme A, free carnitine, and combinations thereof. In an embodiment, the nutritional composition includes free carnitine.

In an embodiment, the-nutritional composition is in an administrable form such as pharmaceutical formulations, nutritional formulations, tube-feed formulations, dietary supplements, functional foods, beverage products or a combination thereof.

In another embodiment, the present disclosure provides methods of selecting a nutritional composition for administration to a patient. The methods include providing a protein. selected from the group consisting of whey, chicken, corn, caseinate, wheat, flax, soy, carob, canola, pea or combinations thereof, and combinations of select minerals including, but not limited to, Mg, Ca, K, and P. Using a conventional method, the acid content of the nutritional composition can be estimated using the PRAL equation: acid content=[(P×0.0366)+(protein (g/day)×acid potential of the protein(s) (mEq/100g protein))+(Cl×0.0268)), and calculating a base content of the nutritional composition using the equation: base content=[(Ca×0.0125)+(Mg×0.0263)+(K×0.0211)+(Na×0.0413)]. The methods include subtracting the base content from the acid content to obtain a PRAL value and selecting the nutritional composition for administration to the patient if the PRAL value is negative. Because we suspect that P is beneficial for the patient (but appears on the acid side of the PRAL equation), this nutrient will be accounted for separately. The final PRAL is reduced using minerals such as P, Ca, K, and Mg.

In an alternative embodiment, the present disclosure provides methods of administering a nutritional composition to a patient in need of same. The methods include providing a protein selected from the group consisting of whey, chicken, corn, caseinate, wheat, flax, soy, carob, pea, canola, cottonseed, potato, rice, egg or combinations thereof, and combinations of select minerals including, but not limited to, Mg, Ca, K, and P, calculating an acid content of the nutritional composition using a modification of the equation stated above, wherein acid content=[(P×0.0366)+(protein (g/day) x acid potential of the protein (mEq/100 g protein))+(Cl×0.0268)], and calculating a base content of the nutritional composition using the equation: base content=[(Ca×0.0125)+(Mg×0.0263)+(K×0.0211)+(Na×0.0413)]. The methods also include subtracting the base content from the acid content to obtain a PRAL value, and administering the nutritional composition to the patient if the PRAL value is negative. The final PRAL is reduced using minerals such as P, Ca, K, and Mg.

In yet another embodiment, computer implemented processes for determining a PRAL value are provided. The processes include providing a computer having an input device and a computer processor so constructed and arranged to calculate the metabolic acid potential of a nutritional composition using a modified PRAL equation to account for P and Na correctly. The protein is selected from the group consisting of whey, chicken, corn, caseinate, wheat, flax, soy, carob, pea or combinations thereof. In an embodiment, the PRAL value is negative. The mineral content of the formulations is manipulated to reduce the PRAL.

In still yet another embodiment, the present disclosure provides methods for preserving and/or preventing bone loss as well as skeletal muscle mass. The methods include providing a protein and combinations of select minerals (e.g., Mg, Ca, K, P) selected from the group consisting of whey, chicken, corn, caseinate, wheat, flax, soy, carob, pea, canola, cottonseed, potato, rice, egg or combinations thereof, calculating an acid content of the nutritional composition using the modified PRAL equation.

In another embodiment, methods for buffering acidosis in a patient in need of same are provided. The methods include providing a protein selected from the group consisting of whey, chicken, corn, caseinate, wheat, flax, soy, carob, canola, pea or combinations thereof, calculating an acid content of a nutritional composition using the modified PRAL equation.

An advantage of the present disclosure is to provide an improved tube feed formulation with net alkaline load that promotes renal health

Yet another advantage of the present disclosure is to provide a nutritional composition that promotes bone health.

Still yet another advantage of the present disclosure is to provide nutritional compositions that preserve skeletal muscle mass.

Another advantage of the present disclosure is to provide a method of administering a nutritional composition.

Another advantage of the present disclosure is to improve clinical patient outcomes, the functional mobility of patient and enhance the quality of life.

Yet another advantage of the present disclosure is to provide a computer implemented process for determining a PRAL of a nutritional composition.

Additional features and advantages are described herein, and will be apparent from the following Detailed Description.

### BRIEF DESCRIPTION OF THE FIGURES

FIG. 1 shows a graph demonstrating the relationship between net acid excretion (“NAE”) prediction and bone mineral density (“BMD”).

### DETAILED DESCRIPTION

The notion that dietary protein increases calcium loss began in the early twentieth century and was later formulated into a hypothesis. The underlying mechanism for this hypothesis is based on the role of bone as a buffering reservoir that aids the kidneys and lungs in the tight regulation of the systemic hydrogen ion concentration. Dietary practices which lead to chronic production of acid ash, such as diets high in protein and phosphates, are hypothesized to tap into this alkali reservoir and cause a gradual dissolution of bone mineral and as such are considered a risk for hypercalciuria and osteoporosis. The increased endogenous acid production is thought to also increase glomelular filtration rate and thus decrease the renal reabsorption of calcium leading to increased urinary calcium and bone loss. Conversely, foods such as fruits can be acid containing but are net alkaline producing in nature which can positively impact acid-base balance. Remer and Manz have developed a calculation to estimate the potential renal acid in which anions such as phosphate, sulfate and chloride are classified as “acidic” ions while cations namely sodium, potassium, calcium and magnesium have been classified as “alkaline.” Based on the calculation, because of the high sulfate and phosphate content, meat, fish, dairy and grains are considered to be detrimental to bone health; while high potassium containing foods, like fruits and vegetables, are thought to be protective to bone health. Recent evidence from intervention studies investigating the role of dietary protein and phosphate do not support the acid-ash hypothesis and will be discussed in more detail below. Paradoxically, the formula assigns sodium a protective role for calcium balance. However, sodium has been shown to compete with calcium for renal re-absorption and thus may impair calcium retention. Both salt-loading studies and reports of free-living populations find that urinary calcium excretion increases approximately 1 mmol (40 mg) for each 100 mmol (2300 mg) increase in dietary sodium in normal adults. The nutritional compositions and formulations of the present disclosure do not increase sodium content to increase alkalinity of a consumer's diet.

### Dietary Protein

Consistent with the acid-ash hypothesis, the hypercalciuric effect of sulfates has been demonstrated in studies using both purified and common sources of protein when phosphorus intakes were held constant. However, when increased protein is added as common foods, without manipulation of the phosphorus content, hypercalciuria is not observed. Although the sulfur amino acids are thought to cause hypercalciuria, the high phosphorus content of these proteins has been found to negate this effect. Many staple plant proteins, such as wheat and rice, have sulfur amino acid contents that are similar to or higher than meats but the co-existing alkalis are thought to reduce the dietary acid load.

Furthermore, the increased ammoniagenesis observed with higher protein intake may partly neutralize the acid production. Furthermore, high protein intake may increase intestinal calcium absorption. Thus, the net effect of a protein source on calcium balance is determined by many co-existing factors both in the protein source as well as the whole diet and is therefore difficult to predict.

### Benefits of Dietary Protein on Calcium Metabolism

Recent studies using stable isotope, whole body methodology and carefully controlled diets of several weeks duration have shown that increased protein intake does not adversely affect whole body calcium retention or any indices of bone metabolism. Also, moderately high protein intake (e.g., ˜20% of energy) reduced markers of bone resorption (urinary deoxypyridinoline) and increased serum insulin-like growth factor (IGF-1) without affecting PTH. It was concluded that under practical dietary conditions, increased dietary protein was not detrimental to calcium balance or bone health. In fact, rather than an antagonistic effect, the findings also indicated a synergistic interaction between dietary protein and calcium such that a high protein intake increased calcium absorption when calcium intake was low (e.g., ˜600 mg/d). This beneficial effect of high protein intake may be in part due to the higher phosphate intake which accompanied the higher protein intake. This notion is strongly supported by a recent meta-analysis of 12 studies (including 269 subjects) in which Fenton and colleagues quantified the contribution of phosphate intake to bone loss in healthy adults. The data indicated that urinary calcium loss decreases in response to phosphate intake, independent of calcium intake or form of phosphorus. See, Fenton et al., “Phosphate decreases urine calcium and increases calcium balance: a meta-analysis of the osteoporosis acid-ash diet hypothesis,” Nutrition J., 8:41 (2009).

The benefits of increased phosphate may be particularly beneficial for severely ill patients who are characterized by increased risk of infection as a result of metabolic alterations resulting from the inflammatory response. In this patient population, the host status dictates the response and virulence of microbes. Low intestinal concentrations of phosphate have been .shown to turn on microbial virulence, while high phosphate turns off quorum sensing, or intercellular signaling between microbes. Extracellular phosphate has been shown to be depleted following acute surgical injury. Intestinal phosphate levels play a role in risk for infection in critically ill patients. See, Long et al., “Depletion of Intestinal Phosphate following Surgical Injury Activates the Virulence of P. aeruginosa causing Lethal Gut-Derived Sepsis,” Surgery, 144:189-197 (2008); Zaborin et al., “Red death in Caenorhabditis elegans caused by Pseudomonas aeruginosa PAO1,” PNAS 1009;106:6327-6332 (2009). If dietary phosphate, or a phosphate analog, is found to play a role in increasing extracellular phosphate levels or intestinal concentrations of phosphate, then a nutritional formulation with increased protein and therefore increased dietary phosphate levels may have a dual benefit for bone health as well as decreasing infection risk in severely ill patients.

While the effects of dietary protein on bone health has been primarily focused on the acid-base equilibrium and the effect on urinary calcium loss, recent evidence does not support this connection. A recent meta-analysis by Fenton and colleagues found that despite a significant linear relationship between an increase in Net Acid Excretion (“NAE”), which is a measure of acid in the urine (NAE=titratable acid+NH₄⁺−HCO₃⁻), and urinary calcium, there was no relationship between changes in NAE and markers of bone breakdown (e.g., urinary N-telopeptides). They concluded that evidence from high quality studies do not support the concept that the calciuria associated with higher NAE reflects a net loss of whole body calcium and or that increasing the diet acid load promotes skeletal bone mineral loss or osteoporosis. The accumulated evidence indicates that the effects of changes in urinary calcium may have been overemphasized in determination of the effect of dietary protein on body calcium balance and therefore bone health in those consuming mixed, varied diet. However, in the case of tube fed patients, it is possible that even a small net acid load can be detrimental over time.

There is ample evidence indicating that increased dietary protein has favorable systemic effects beyond its effect on calcium excretion. Experimental and clinical studies suggest that protein intake affects both the production and action of growth factors such as IGF-1. It is well established that a decreased serum concentration of IGF-1 is strongly associated with decreased bone strength in animals and an increase in risk of osteoporotic fractures in humans. Both the hepatic production and the total level of IGF-1 are under the influence of dietary proteins and protein restriction has been shown to reduce plasma IGF-1 in humans inducing a resistance of target organs to the action of growth hormone. In a controlled, 1-year intervention study, 20 g of supplemental dietary protein/d improved hip bone mineral density (BMD) (and serum IGF-1) in elderly patients with recent hip fracture.

There may be a clinical application for patients receiving long term tube feeding formulas made with purified proteins. For example, a 2-year randomized, controlled trial, the longest alkali supplementation trial to date, potassium citrate supplementation did not affect bone turnover or BMD, indicating that any benefit of fruit and vegetable intake cannot be explained by the potassium intake alone.

A close examination of the evolution of our understanding of the acid-ash hypothesis and the role of protein in bone health points to the following:

1) The conventional scientific reductionism in which the effect of protein has been reduced to its sulfur amino acids (not accounting for the accompanying phosphorus) and in which urinary calcium has been used as an indicator of net effect on bone health (ignoring variations in calcium absorption and systemic effects of dietary protein) has led to public advice negated by the current body of evidence.

2) Because we consume complex foods and not isolated nutrients, and because human health is a system of organs with complex interrelationships and dynamic adaptive capacity, the study of the effect of whole foods on human health demands complex design and the scientific will to tolerate multiple variables. Nutritionists and registered dietitians are uniquely poised to identify scientific questions of public health relevance, help formulate hypotheses intended to test the effects of whole foods on whole systems and to generate relevant, substantiated public health advice.

Patients that are either inactive or fed one single diet with a relatively high acid potential for an extended length of time are susceptible to metabolic disturbances. For example, long-term tube-fed patients may suffer from such disturbances. Although the nutritional needs of the patient may be met through, for example, tube feeding, the current formulas are not optimized for maintenance of the patient's acid-base status over long periods.

Patients who receive long-term tube feeds often remain on a single dietary source for weeks, months, or even years. While the body's blood pH is fairly well maintained over time, primarily through regulation by the kidneys and lungs, dietary intake can significantly influence the body's acid/base balance. Hospitalized, institutionalized, and recovering patients may be at an increased risk of metabolic disturbances caused by poor renal and/or pulmonary function. As a result, the acid-base potential of the diet becomes increasingly important in maintenance of the patient's health, including musculoskeletal and immune health.

Upon ingestion and after metabolism, foods can be categorized as either net acidic versus net alkaline producing. For example, “acid-ash” and “alkaline-ash” diets have been traditionally defined as the balance between anions (Cl, P, S) and cations (Na, K, Mg, Ca). However, increased P has been shown to reduce urinary calcium loss; The acid-ash diet, or more acid producing diet, has an excess of anions over cations (and vice versa for the alkaline-ash diet). Increasingly, acid producing diets have been found to negatively impact musculoskeletal and immune health.

Because long-term tube fed patients lack variation in their food sources they may be particularly susceptible to the effects of acid-forming diets. Although the kidneys are efficient at neutralizing acids, long term exposure to high acid is believed to overwhelm the kidneys’ capacity to neutralize acid and potential damage may occur. As a result, alkaline compounds that include, but are not limited to, calcium are used to neutralize these dietary acids (in the case of skeletal muscle, glutamine can act as a buffer). The most readily available source of calcium in the body is bone. One theory is that high acid diets may contribute to bone loss as the body mobilizes stores of calcium to buffer metabolic acid. The hypothesis is that low acid diets may result in'benefits that include attenuation of bone and skeletal muscle loss as well as maintaining renal health. See, Wachman, A., et al., “Diet and Osteoporosis,” Lancet, 1:958-959 (1968); see also Frassetto L, et al., “Potassium Bicarbonate Reduces Urinary Nitrogen Excretion in Postmenopausal Women,” J. Clin. Endocrinol. Metab., 82:254-259 (1997).

Some individuals may receive all or part of the nutritional requirements from formulated or synthetic diets. Dietary intake may include 50-100% of their nutrient needs through supplemental formulas, where the range maybe oral or tube feeding. Reasons for partial to complete supplementation of the diet with specialized formulas include institutional or home care and conditions such as chronic obstructive pulmonary (“COPD”) patients who have difficulty consuming an adequate diet as a result of their physical and/or psychological limitations (e.g., fatigue, fear of choking or suffocation during chewing or swallowing, and increased energy needs); patients having undergone major surgery, whose energy and protein needs are increased and are unable to ingest adequate protein or nutrients with a normal diet; individuals suffering from a neuromuscular disease, such as Amyotrophic Lateral Sclerosis (“ALS”), where the majority of the diet may come from supplementation with tube feeding and oral intake is reserved for pleasure; ageing care patients that have dietary restrictions or physical, economic or social conditions that limit their ability to consume an adequate diet; pediatric patients, such as cystic fibrosis, where dietary supplementation is administered overnight via tube feeding to meet their nutritional requirements; head and neck cancers where oral intakes are not possible and tissue damage to the gastrointestinal tract results in direct-gastric tube feeding, as well as other catabolic conditions in which individuals cannot meet their nutritional requirements for a variety of reasons.

Various measurements have been utilized to measure acidity versus alkalinity after metabolism of nutritional compositions. Measurements that rely on physiological markers include NAE, as discussed above. Because the NAE is determined by adding up the urinary acidic anions and subtracting out the alkaline cation, it cannot be used to predict the influence of the diet. Therefore, a different technique to approximate the effect of the diet must be used. The most widely accepted theoretical model to approximate the dietary acid or base load is called the potential renal acid load (“PRAL”). The PRAL is represented and measured in milliequivalents of acid (mEq). A calculation for PRAL, as described by Remer and Manz, “Potential renal acid load of foods and its influence on urine pH,” J. Am. Diet Assoc., 95:791-797 (1995), is as follows:

PRAL (mEq/d)=Acid−Base

Acid=[(P×0.0366)+(Protein (g/day)×0.4888)+(Cl×0.0268)]

Base=[(Ca×0.0125)+(Mg×0.0263)+(K×0.0211)+(Na×0.0413)]

In the formula, P is the phosphorous content of the foodstuff (mg/day), Cl is the chloride content of the foodstuff (mg/day), Ca is the calcium content of the foodstuff (mg/day), Mg is the magnesium content of the foodstuff (mg/day), K is the potassium content of the foodstuff (mg/day) and Na is the sodium content of the foodstuff (mg/day). Values for the variables in the equations (e.g., the content of P, Cl, Ca, etc. in foodstuffs) may be obtained, for example, from any commercial dietary analysis software program such as, for example, Food Processor by ESHA Research or Nutritionist Pro™ Diet Analysis by Axxya Systems LLC. Similarly, the values for the variables may also be found on the United States Department of Agriculture's National Nutrient Database for Standard Reference at http://www.nal.usda.gov/fnic/foodcomp/search/. A simplified approach to estimate net acid production is the Protein to Potassium ratio. Increased values correlated with measures of renal net acid excretion (RNAE). See, Frassetto L A et al., “Estimation of net endogenous noncarbonic acid production in humans from diet potassium and protein contents,” Am. J. Clin. Nutr. (1998).

The single largest contributor to the acid-base potential of the nutritional composition is protein, but the generic term “protein” does not distinguish between the different sources of protein, which can have very different impacts on the diet's acid-base balance. These differences have not previously been accounted for in equations used for predicting acid influence of compositions after metabolism. Indeed, the “protein” in the Remer and Manz equation is simply the amount of protein in the composition, regardless of the type of protein used or whether a mixture of different proteins was used.

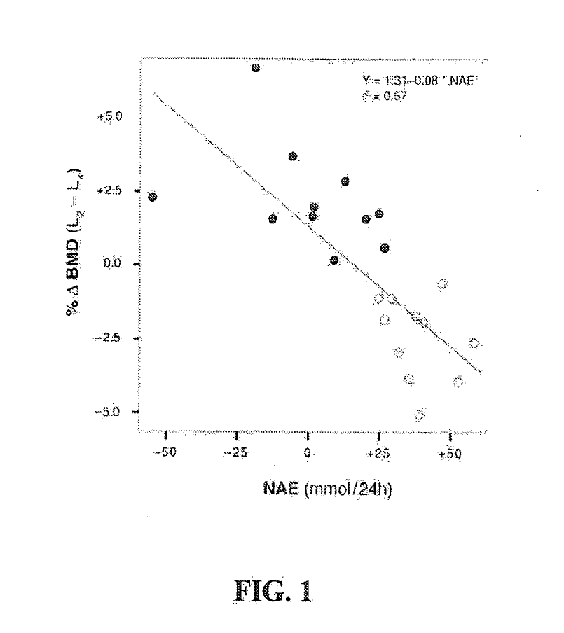

With regard to bone health, data from a meta-analysis of 25 studies supports the conclusion that acid producing diets may negatively impact musculoskeletal health. Indeed, there was a significant relationship found between NAE and calcium excretion (bone mineral density (“BMD”), as is illustrated in FIG. 1. See, Fenton T R, et al., “Meta-analysis of the quantity of calcium excretion associated with the net acid excretion of the modern diet under the acid-ash diet hypothesis,” Am. J. Clin. Nutr., 88:1159-1166 (2008); see also, Jehle, S., et al., “Partial Neutralization of the Acidogenic Western Diet with Potassium Citrate Increases Bone Mass in Postmenopausal Women with Osteopenia,” J. Am. Soc. Nephrol., 17: 3213-3222 (2006). Similarly, Jajoo et al. found that renal NAE was associated with urinary calcium excretion, PTH levels and urinary N-telopeptide (a marker of bone breakdown). See, Jajoo R, et al., “Dietary acid-base balance, bone resorption, and calcium excretion,” J. Am. Coll. Nutr., 25:224-230 (2006).

Using the measure of dietary PRAL, Alexy et al. reported a correlation between high dietary PRAL and lower cortical area and bone mineral content in children. See, Alexy U, et al., “Long-term protein intake and dietary potential renal acid load are associated with bone modeling and remodeling at the proximal radius in healthy children,” Am. J. Clin. Nutr., 82:1107-1114 (2005). Additionally, young girls consuming high amounts of fruits, an alkaline producing food, had high heel bone mineral density. See, McGartland C P, et al., “Fruit and vegetable consumption and bone mineral density: the Northern Ireland Young Hearts Project,” Am. J. Clin. Nutr., 80:1019-1023 (2004).

According to the PRAL calculation, high protein diets push the acid-base balance towards acidic as a result of their sulfur amino acids. However, there exists controversy about whether this effect of dietary protein is anabolic or catabolic on bone. Purified dietary proteins (such as whey isolate, caseinates, etc., as used in most enteral nutrition formulations) are traditionally considered to increase urinary calcium excretion. See, Schuette S A, et al., “Studies on the mechanism of protein-induced hypercalciuria in older men and women,” J. Nutr., 110:305-315 (1980); see also Allen L H, et al., “Protein-induced hypercalciuria: a longer term study,” Am. J. Clin. Nutr., 32:741-749 (1979). Other research shows that a diet higher in whole protein may be beneficial when the diet is low in calcium. See, Hunt J R, et al., “Dietary protein and calcium interact to influence calcium retention: a controlled feeding study,” Am. J. Clin. Nutr., 89:1357-1365 (2009). Addint to the controversy is the systemic effects of the anabolic IGF response and the potential protective effect of P that comes with animal proteins.

As mentioned previously, the physiological measurement NAE is a good estimate for endogenous acid production and correlates inversely with changes in bone mass. In a 12-month study, NAE was correlated to decreased BMD. See, Jehle et al., 2006. In addition, an increase in BMD was observed following prolonged alkali administration. However, the effect on bone formation has yet to be elucidated. Observed increases in BMD may be more related to anti-resorption than bone formation. Therefore, bone mineral density may be improved by the use of specific tube-fed formulas having specific acidities.

In addition to bone specific effects, human correlational data suggests that dietary intakes of fruits and vegetables support a net alkaline environment which can help regulate metabolic homeostasis. This net alkaline state has been associated with an enhanced preservation of lean body mass in older adults. See, Dawson-Hughes et al., “Alkaline diets favor lean tissue mass in older adults,” Am. J. Clin. Nutr., March; 87(3):662-5 (2008). Thus, the manipulation of P, Na, Mg, K and Ca in complete nutritional formulas can serve to enhance net alkaline production to further minimize endogenous skeletal muscle proteolysis as well as preserve lean body mass. The form of these minerals provided in nutritional formulas may impact net alkaline production.

The cell energy charge has been proposed as an important control for the cell to favor either anabolic or catabolic processes. Metabolic stress, nutritional stress, or both may result in a loss of nucleotides from the adenylate pool and become conditionally essential under these conditions. The maintenance of the cell energy charge can attenuate the upregulation of catabolic processes resulting from metabolic stress, nutritional stress, or both which includes protein breakdown.

Other mechanisms involved in protein degradation include, for example, ubiquitin (“Ub”), which functions to regulate protein turnover in a cell by closely regulating the degradation of specific proteins, calpain (the calpain family of proteases consists of 3 well-characterized proteins, g-calpain, m-calpain and calpastatin) and lysosomal (organelles containing digestive enzymes). AMP Protein Kinase (“AMPK”) is a protein that serves as a cell energy charge sensor that responds to ATP/AMP as well phosphocreatine/creatine (“PCr”/“Cr”) changing ratios for the prioritization of cellular processes based on available energy. Specifically, AMPK can target the translational control of skeletal muscle protein synthesis as well as upregulate the ubiquitin proteosome pathway.

Further, metabolic acidosis has been described for its association with skeletal muscle wasting in several different conditions (e.g., chronic renal failure; obese on weight loss diets) and has been the subject of review. See, Caso G, et al., “Control of muscle protein kinetics by acid-base balance,” Curr. Opin. Clin. Nutr. Metab. Care, 8:73-76 (2005). During acidosis, skeletal muscle proteolysis appears to be an adaptive response. Glutamine breakdown from skeletal muscle is a substrate for ammonia which can accept protons and possibly reduce acidosis. Ammonium liberated during glutamine deamination (loss of 1 of 2 ammonia group of glutamine) facilitates the excretion of acids by accepting a proton which may help to minimize acidosis. As the kidney increases extraction of glutamine there is a need to release more glutamine from skeletal muscle and liver, as well as decreased utilization in the intestine. This can have negative consequences including loss of lean skeletal muscle mass and reduced glutamine availability for the rapidly proliferating intestinal cells, which may negatively impact immune function. See, Wellbourne et al., “The Glutamine/Glutamate Couplet and Cellular Function,” News in Physiological Sciences, 16(4):157-160 (2001). Correction of acidosis may help to preserve skeletal muscle mass, improve glucose tolerance, enhance functional mobility and improve the health of patients with pathological conditions associated with acidosis.

Glutamine has other roles in the body. One is as a precursor of arginine via citrulline. The addition of exogenous citrulline may spare muscle protein breakdown since citrulline may conserve glutamine and allow for more glutamine to serve as a proton acceptor. This diagram shows how the addition of citrulline can block the conversion of ornithine to citrulline. Additionally with citrulline present to serve as the precursor to arginine, the higher level of arginine would also allow for a larger portion of the ornithine to come from arginine and not glutamine.

Chronic low grade metabolic acidosis can occur when dietary intake of foods metabolized to acid exceeds the intake of foods metabolized to base. In a study including post-menopausal women on a high protein diet (acid producing), consumption of potassium bicarbonate decreased net rates of endogenous acid production and total urinary nitrogen levels decreased. See, Frassetto, 1997. More recently, KHCO₃ supplementation was shown to slow nitrogen loss in older adults. See, Ceglia L, et al., “Potassium bicarbonate attenuates the urinary nitrogen excretion that accompanies an increase in dietary protein and may promote calcium absorption,” J. Clin. Endocrinol. Metab., 94:645-653 (2009).

Additionally, urinary potassium excretion (a marker of dietary potassium intake) was correlated to percent of lean body mass. See, Dawson-Hughes et al., “Alkaline diets favor lean tissue mass in older adults,” Am. J. Clin. Nutr., March; 87(3):662-5 (2008). It was concluded that this nitrogen sparing is “potentially sufficient to both prevent continuing age-related loss of skeletal muscle mass and restore previously accrued deficits.” Id.

In addition to both bone and skeletal muscle health, optimization of nutritional compositions may also support renal health, which can be negatively influenced by metabolic acidosis (chronic or acute). Specifically, metabolic acidosis can influence hormones that control fluid balance in the body. Fluid balance is also responsible for mineral excretion (electrolytes) that are key in maintenance of acid-base balance.

In order to buffer “acidosis,” dietary glutamine and dietary citrulline may be used. With respect to glutamine, for example, during a state of acute or chronic acidosis, skeletal muscle breakdown appears to be an adaptive response partly driven by the need for glutamine. See, Epler et al. “Metabolic acidosis stimulates intestinal glutamine absorption,” J. Gastro. Surg. (2003). Glutamine available for proton quenching comes from only two sources: the diet and the skeletal muscle. Chronic skeletal muscle catabolism is highly undesirable can lead to skeletal muscle atrophy. Glutamine quenches protons (hydrogen) that may be upset in conditions such as chronic obstructive pulmonary disease and renal insufficiency caused by ageing or disease.

Alkaline diets may also be used to offset respiratory insufficiency, as discussed above. For example, in the Intensive Care Unit (“ICU”), patients often require artificial respiration. This condition results in proton build up because the individuals cannot naturally increase their breathing rate to ‘blow off’ excess carbon dioxide and protons and may lead to metabolic acidosis. Therefore, it would be beneficial to use glutamine in combination with the optimized alkaline formula compositions of the present disclosure for both tube feeding and parenteral administration. Indeed, correction of acidosis may help to preserve skeletal muscle mass and improve the health of patients with pathological conditions associated with acidosis. Additionally, patients in the ICU typically have a high demand for, but a low level of glutamine.

Further, a shunt of glutamine for correcting acidosis also contributes to immunosuppression as the glutamine supply to the enterocytes of the gut is reduced. Therefore, supplementing and correcting metabolic acidosis may also improve the patient's immune status.

In addition to glutamine, dietary citrulline may also be used as a buffer of “acidosis.” As previously discussed, the NAE equation is used to determine the overall load of acid in the body. Because the NAE is equal to the amount of titratable acid and ammonium in the urine minus the bicarbonate (e.g., NAE=((Titratable acid+NH₄+)-bicarbonate)) it would be desirable to reduce the amount of circulating nitrogen not bound in amino acids. Citrulline has one less nitrogen than arginine and may be substituted for arginine.

The oxidation of dietary fatty acids and hepatic desaturation/elongation of palmitic acid can occur to a greater degree in abdominally obese individuals. This increased oxidation may represent a compensatory mechanism to redirect fatty acids from incorporation into the liver to prevent liver fat accumulation. However, under conditions of metabolic acidosis, reduced levels of free coenzyme A and free carnitine may limit the carnitine-mediated transfer of long-chain fatty acids into mitochondria for oxidation. Thus, under conditions of metabolic acidosis obese individuals may be more susceptible to liver fat accumulation whereas the present alkaline formula would seek to attenuate or minimize such a response. This metabolic improvement could improve the preservation of lean body mass. The inefficient mobilization of adipose tissue energy stores in patients leads to the need to feed high protein to reserve lean body mass. Better functioning of the system may help preserve muscle and/or reduce the need for feeding a very high level of protein. As such, in an embodiment, the nutritional compositions of the present disclosure may include free coenzyme A, free carnitine, or combinations thereof. In an embodiment, the nutritional compositions include free carnitine. In an embodiment, the nutritional compositions include from about 1 to about 220 mg of free carnitine per complete feed. In another embodiment, the nutritional compositions include from about 100 to about 200 mg of free camitine per complete feed.

Moreover, selection of dietary protein sources that minimize the diet's acid potential is also expected to have an additional benefit of maintaining insulin-like growth factor-1 (IGF-1) and its binding proteins (IGFBPs). The anabolic growth factor IGF-1 is attenuated in persons with renal insufficiency (disease and ageing). Therefore, selections of protein(s) that can be fed in higher concentrations, but that contribute the least sulfur amino acid and thus contribute less to the acid status are beneficial.

As discussed above, the most widely accepted theoretical model to approximate the dietary acid or base load in the body after metabolism of a nutritional composition is the PRAL calculation by Remer and Manz. However, this method is not precise. The effect of protein on the total acid potential in the Remer and Manz equation' is generic and takes into account only the amount of protein used, regardless of the type(s) of protein. As such, the Remer and Manz equation does not reflect the varying contribution that is made by different protein sources, which inherently have different acid potentials and may be provided in varying amounts in a composition. In contrast, Applicant has found that by more precisely determining the acid component of the PRAL equation, the acid-base potential of a nutritional composition may be more accurately and easily predicted to enable better formula development.

Specifically, Applicant has found that taking into consideration the type of protein, which contains an inherent amount of the sulfur-containing amino acids methionine and cystine, calculating the molar amounts of each amino acid in the protein, and using these values to determine the molar amount of sulfur in the protein, a more accurate acid potential for the protein component of a foodstuff may be determined using a modified version of the PRAL equation set forth above. For example, the acid potential of the nutritional compositions of the present disclosure may be obtained by substituting the “Protein (g/day)×0.4888” value in the Remer and Manz acid equation with “protein (g/day)×acid potential of the protein (mEq/100 g protein).” Accordingly, the improved equation for determining PRAL values is as follows:

PRAL (mEq/d)=Acid−Base

Acid=[(P×0.0366)+(protein (g/day)×acid potential of the protein (mEq/100 g protein))+(Cl×0.0268)]

Base=[(Ca×0.0125)+(Mg×0.0263)+(K×0.0211)+(Na×0.0413)]

Similar to the Remer and Manz equation, in the improved equation, P is the phosphorous content of the foodstuff (mg/day), Cl is the chloride content of the foodstuff (mg/day), Ca is the calcium content of the foodstuff (mg/day), Mg is the magnesium content of the foodstuff (mg/day), K is the potassium content of the foodstuff (mg/day) and Na is the sodium content of the foodstuff (mg/day). However, Applicant's improved equation now takes into consideration the acid potential of specific protein sources.

As previously discussed, the single largest contributor to the acid/base potential of a nutritional composition is protein due, at least in part, to the sulfur amino acid content, which varies with each different type of protein. The two primary amino acids that are found in proteins and which contain sulfur are methionine and cystine. To calculate the acid potential of each individual protein, the amount of grams of methionine per 100 grams of protein, and the amount of grams of cystine per 100 grams protein is required. From the amounts of methionine and cystine, molar amounts of each may be calculated using each respective molar mass. The molar mass of methionine is 149.2 g/mol and the molar mass of cystine is 240.3 g/mol. The molar amount of sulfur may then be calculated using the following equation:

mmol Sulfur (mEq/diet)=(mg methionine/149.2 g/mol)+(2×(mg cystine/240.3 g/mol)).

To obtain the acid potential of the protein, the molar amount of sulfur is multiplied by 2. For example, the acid potential of whey protein is calculated as follows:

Sulfur (mEq/diet)=2×[(2200 mg methionine/149.2 g/mol)+(2×(2400 mg cystine/240.3 g/mol))].

Table 1 provides several additional acid-potentials for various protein sources based on the sulfur amino acid content.

TABLE 1

| | | | Methionine: | Cystine: | | | | |
|---|---|---|---|---|---|---|---|---|
| | g/100 g protein | | 149 g/mole | 240.3g/mole | | | | |
| Type | Met | Cystine | mmol | | mmol Sulfur | | total mmol S | mEq |
| Eqg protein | 2.6 | 3.4 | 17.450 | 14.149 | 17.450 | 28.298 | 45.748 | 91.495 |
| Whey | 2.2 | 2.4 | 14.765 | 9.988 | 14.765 | 19.975 | 34.740 | 69.480 |
| Cottonseed protein | 2.6 | 1.6 | 17.450 | 6.658 | 17.450 | 13.317 | 30.766 | 61.533 |
| Chicken | 2.7 | 1.4 | 18.121 | 5.826 | 18.121 | 11.652 | 29.773 | 59.546 |
| Micellar casein | 0.7 | 2.8 | 4.698 | 11.652 | 4.698 | 23.304 | 28.002 | 56.004 |
| Potatoe protein | 0.9 | 2.5 | 6.040 | 10.404 | 6.040 | 20.807 | 26.848 | 53.695 |
| Corn | 2.1 | 1.4 | 14.094 | 5.826 | 14.094 | 11.652 | 25.746 | 51.492 |
| Rice protein | 1.2 | 2 | 8.054 | 8.323 | 8.054 | 16.646 | 24.700 | 49.399 |
| Caseinate | 2.9 | 0.4 | 19.463 | 1.665 | 19.463 | 3.329 | 22.792 | 45.585 |
| Wheat protein | 1.4 | 1.6 | 9.396 | 6.658 | 9.396 | 13.317 | 22.713 | 45.425 |
| Flax protein | 1.3 | 1.5 | 8.725 | 6.242 | 8.725 | 12.484 | 21.209 | 42.418 |
| Flax | 1.3 | 1.4 | 8.725 | 5.826 | 8.725 | 11.652 | 20.377 | 40.754 |
| Canola protein | 0.6 | 1.9 | 4.027 | 7.907 | 4.027 | 15.814 | 19.840 | 39.681 |
| Soy | 1.3 | 1.3 | 8.725 | 5.410 | 8.725 | 10.820 | 19.545 | 39.089 |
| CGMP+ | 0.5 | 1.8 | 3.356 | 7.491 | 3.356 | 14.981 | 18.337 | 36.674 |
| Carob | 1 | 1.35 | 6.711 | 5.618 | 6.711 | 11.236 | 17.947 | 35.895 |
| CGMP | 0 | 1.9 | 0.000 | 7.907 | 0.000 | 15.814 | 15.814 | 31.627 |
| Pea | 1.1 | 1 | 7.383 | 4.161 | 7.383 | 8.323 | 15.705 | 31.411 |

Thus, according to the improved equation, for each 100 g protein, or fraction thereof, from the protein sources shown in Table 1 the total daily diet acidity (expressed in milli-equivalents acid or mEq) can be calculated. If a product is formulated entirely from whey protein then this number (e.g., 100 g whey protein isolate=69.48 mEq acid) would replace the generic calculation of protein in the Remer and Manz equation where acidity (mEq) of the “protein” is expressed as grams of protein per day×0.4888. Similarly, using the improved equation, 50 g of whey protein×69.48 mEq/100 g protein=34.74 mEq acid. This clearly illustrates the difference in acidity of the protein when the acid potential of the specific protein is considered (34.74 mEq) as opposed to when 50 g of a generic “protein” is used (50 g×0.4888=24.44 mEq acid). Accordingly, a protein with lower acid potential (e.g., pea or soy protein isolate) would result in a lower contribution to the total acid balance than the generic calculation for protein.

Assuming an absorption of 75% of the orally ingested protein, the final values in the right-most column of Table 1 (mEq) should be multiplied by a factor or 0.75 to account for the absorption.

In addition to the use of single, specific types of proteins, the acidity of blends of proteins can also be easily determined by the improved equation by using the fractional contribution of each protein source according to its sulfur amino acid content. Therefore, use of the improved equation allows for the preparation of nutritional compositions having several

Further, from these calculations, it appears that lower PRAL diets (more alkaline producing versus more acid producing) may have beneficial effects on musculoskeletal, immune and renal health. Nestle's blenderized tube feeds, Compleat® and Isosource® Mix, already have a low calculated PRAL value. However, these formulas can be further optimized to deliver greater benefits. The long term use of these optimized tube feeds may be appropriate for the maintenance of bone, skeletal muscle mass and strength, and renal or pulmonary function. Populations expected to benefit include long-term home care patients, elderly, ICU patients, pediatric patients requiring medical nutrition, bed-ridden patients, chronic obstructive pulmonary disease (“COPD”) patients, ventilated patients, patients recovering from trauma, diabetic patients, hepatic patients, patients with renal insufficiency, etc.

The calculations for the modified equation may be performed manually by a user or generated automatically using a computer implemented process. For example, computers having a processor can be used to estimate the acidity of the nutritional compositions. The processor should be so constructed and arranged to be able to calculate an acid component of the improved PRAL equation using the already cited modified PRAL equation.

Accordingly, use of the improved modified equation of the present disclosure to make and/or use nutritional compositions provides several benefits. For example, the improved equation and methods of using the equation accurately predict the physiological response to Phosphorus and Sodium in a patient's diet. Further, the improved equations provide a user the ability to formulate a diet that minimizes the impact of acid/base potential of a patient's diet on the patient. Moreover, consumption of the nutritional compositions derived via use of the improved equations provide resultant clinical benefits to the patient's musculoskeletal health including, but not limited to, preservation of lean body mass and bone mineral density.

As used herein, the term “nutritional composition” includes, but is not limited to, complete nutritional compositions, partial or incomplete nutritional compositions, and disease or condition specific nutritional compositions. A complete nutritional composition (i.e., those which contain all the essential macro and micro nutrients) can be used as a sole source of nutrition for the patient. Patients can receive 100% of their nutritional requirements from such complete nutritional composition. A partial or incomplete nutritional composition does not contain all the essential macro and micro nutrients and cannot be used as a sole source of nutrition for the patient. Partial or incomplete nutritional compositions can be used as a nutritional supplement. A disease or condition specific nutritional composition is a composition that delivers nutrients or pharmaceuticals and can be a complete or partial nutritional composition.

Accordingly, the nutritional composition can be a complete feeding or an oral nutritional supplement. As used herein, an “oral nutritional supplement” includes, but is not limited to, orally ingested formulations, enteral nutrition formulations and tube feeds. The nutritional composition can be in a formulation designed for any mammal such as a human or an animal. The key acid or base contributing ingredients in the nutritional composition can also be provided as a modular product. A modular product can be defined as a method of delivering one or more specific nutrients as a supplement and not intended to be used for sole source nutrition. In an embodiment, the nutritional composition is in an administrable form selected from the group consisting of pharmaceutical formulations, nutritional formulations, tube-feed formulations, total parenteral nutrition formulations, enteral nutrition formulations, dietary supplements, functional foods and beverage products.

As used herein, a “tube feed” formulation is preferably a complete or incomplete nutritional product that is administered to an animal's gastrointestinal system, including but not limited to an oral access port, nasogastric tube, orogastric tube, gastric tube, jejunostomy tube (J-tube), percutaneous endoscopic gastrostomy (PEG), port, such as a chest wall port that provides access to the stomach, jejunum and other suitable access ports.

As used herein, “effective amount” is preferably an amount that prevents a deficiency, treats a disease or medical condition in an individual or, more generally, reduces symptoms, manages progression of the diseases or provides a nutritional, physiological, or medical benefit to the individual. A treatment can be patient- or doctor-related. In addition, while the terms “individual” and “patient” are often used herein to refer to a human, the present disclosure is not so limited. Accordingly, the terms “individual” and “patient” refer to any animal, mammal or human having or at risk for a medical condition that can benefit from the treatment.

In an embodiment, the nutritional compositions comprise a source of protein. The protein source may be dietary protein. The dietary protein is any suitable dietary protein including, but not limited to animal protein (such as milk protein, meat protein or egg protein), vegetable protein (such as soy protein, wheat protein, rice protein, and pea protein), or a combination thereof. In an embodiment, the protein is selected from the group consisting of whey, chicken, corn, caseinate, wheat, flax, soy, carob, pea, canola, cottonseed, potato, rice, egg or combinations thereof. In another embodiment, the protein includes pea protein. Regardless of the protein source, the protein should have low acid potential.

In an embodiment, the PRAL value for a tube feed formulation is between about −20 mEq and about −100 mEq. In another embodiment, the PRAL value for a tube feed formulation is between about −22 mEq and about −95 mEq. In another embodiment, the PRAL value for a tube feed formulation is between about −24 mEq and about −90 mEq. in another −embodiment, the PRAL value for a tube feed formulation is between about −26 mEq and about −85 mEq. In another embodiment, the PRAL value for a tube feed formulation is between about −28 mEq and about −80 mEq. In another embodiment, the PRAL value for a tube feed formulation is between about −29 mEq and about −75 mEq. In another embodiment, the PRAL value for a tube feed formulation is between about −30 mEq and about −70 mEq.

In an embodiment, the Protein:K for a tube feed formulation is between 0.5 (g/mEq) to 1.25 (g/mEq). In another embodiment, the Protein:K ratio is between 0.75 (g/mEq) to 1.2 (g/mEq). In another embodiment, the Protein:K ratio is between 0.9 (g/mEq) to 1.1 (g/mEq).

In an embodiment, the protein is provided in effective amounts to result in nutritional compositions having large negative PRAL values. In an embodiment, the protein is present in the nutritional compositions in amounts between about 1 g and about 200 g. In another embodiment, the protein is present in the nutritional composition in amounts between about 50 g and about 150 g.

Further, because most vegetable proteins (especially pea protein) have low acid potentials (i.e., pea protein isolate=31.411 mEq/100 g protein), use of vegetable proteins in nutritional compositions will result in a composition having a low acid potential. Thus, in an embodiment, the nutritional compositions include pea protein.

In an embodiment, the nutritional compositions comprise a source of carbohydrates. Any suitable carbohydrate may be used in the present nutritional compositions including, but not limited to, sucrose, lactose, glucose, fructose, corn syrup solids, maltodextrin, modified starch, amylose starch, tapioca starch, corn starch or combinations thereof.

In an embodiment, the nutritional compositions include a source of fat. The source of fat may include any suitable fat or fat mixture. For example, the fat source may include, but is not limited to, vegetable fat (such as olive oil, corn oil, sunflower oil, rapeseed oil, hazelnut oil, soy oil, palm oil, coconut oil, canola oil, lecithins, and the like) and animal fats (such as milk fat).

In an embodiment, the nutritional composition further includes one or more prebiotics and/or fiber (soluble and/or insoluble). As used herein, a prebiotic is a selectively fermented ingredient that allows specific changes, both in the composition and/or activity in the gastrointestinal microbiota, that confers benefits upon host well-being and health. Non-limiting examples of prebiotics include fructooligosaccharides, inulin, lactulose, galactool igosaccharides, acacia gum, soyol igosaccharides, xylool igosaccharides, isomaltooligosaccharides, arabinoxylans, gentiooligosaccharides, lactosucrose, glucooligosaccharides, pecticoligosaccharides, resistant starches, sugar alcohols or combinations thereof.

In an embodiment, the nutritional composition further includes one or more probiotics. As used herein, probiotic micro-organisms (hereinafter “probiotics”) are preferably microorganisms (alive, including semi-viable or weakened, and/or non-replicating), metabolites, microbial cell preparations or components of microbial cells that could confer health benefits on the host when administered in adequate amounts, more specifically that beneficially affect a host by improving its intestinal microbial balance, leading to effects on the health or well-being of the host. In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Saccharomyces, Debaromyces, Candida, Pichia, Torulopsis, Aspergillus, Rhizopus, Mucor, Penicillium, Bifidobacterium, Bacteroides, Clostridium, Fusobacterium, Melissococcus, Propionibacterium, Streptococcus, Enterococcus, Lactococcus, Staphylococcus,

In another embodiment, the nutritional composition further includes one or more amino acids. Non-limiting examples of amino acids include Isoleucine, Alanine Leucine, Asparagine, Lysine, Aspartate, Methionine, Cysteine, Cystine, Phenylalanine, Glutamate, Threonine, Glutamine, Tryptophan, Citrulline, Glycine, Valine, Proline, Serine Tyrosine, Arginine, Histidine or combinations thereof.

In an embodiment, the nutritional composition further includes one or more synbiotics, fish oils, and/or phytonutrients. As used herein, a synbiotic is a supplement that contains both a prebiotic and a probiotic that work together to improve the microflora of the intestine. Non-limiting examples of fish oils include docosahexaenoic acid (DHA) and eicosapentaenoic acid (EPA). Non-limiting examples of phytonutrients include those that are flavonoids and allied phenolic and polyphenolic compounds, terpenoids such as carotenoids, and alkaloids including, for example, quercetin, curcumin, limonin, or combinations thereof.

In an embodiment, the nutritional composition further includes antioxidants. Antioxidants are molecules capable of slowing or preventing the oxidation of other molecules. Non-limiting examples of antioxidants include vitamin A, carotenoids, vitamin C, vitamin E, selenium, flavonoids, Lactowolfberry, wolfberry, polyphenols, lycopene, lutein, lignan, coenzyme Q10 (CoQ10), glutathione or combinations thereof.

In another embodiment, the present disclosure provides methods of selecting a nutritional composition for administration to a patient. The methods include providing a protein selected from the group consisting of whey, chicken, corn, caseinate, wheat, flax, soy, carob, pea or combinations thereof, calculating an acid content of the nutritional composition using the modified, PRAL equation.

In an alternative embodiment, the present disclosure provides methods of administering a nutritional composition to a patient in need of same. The methods include providing a protein selected from the group consisting of whey, chicken, corn, caseinate, wheat, flax, soy, carob, pea, canola, cottonseed, potato, rice, egg or combinations thereof, Calculating an acid content of the nutritional composition using the modified PRAL equation.

In yet another embodiment, computer implemented processes for determining a potential renal acid load (PRAL) value are provided. The processes include providing a computer having an input device and a computer processor so constructed and arranged to calculate a metabolic acid potential of a nutritional composition using the modified PRAL equation.

In still yet another embodiment, the present disclosure provides methods for treating and/or preventing bone loss and methods of preserving skeletal muscle mass. The methods include providing a protein selected from the group consisting of whey, chicken, corn, caseinate, wheat, flax, soy, carob, pea or combinations thereof, calculating an acid content of the nutritional composition using the equation.

In another embodiment, methods for buffering acidosis in a patient in need of same are provided. The methods include providing a protein selected from the group consisting of whey, chicken, corn, caseinate, wheat, flax, soy, carob, pea or combinations thereof and manipulating the P and other cations (Mg, Ca, K) to achieve alkaline load.

By using the improved equation and compositions and methods derived from same, the issues associated with skeletal muscle, bone and immune health may be improved in people who are either inactive or fed high-acid diets over long terms. Indeed, the improved equation provides methods of predicting acidity (acid-ash content) of nutritional compositions or diets in order to precisely determine the alkalinity effect of protein and blends of protein in combination with minerals on the skeletal muscle, bone and immune health of patients consuming same.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A nutritional supplement formulation comprising:
a source of fats;
a source of carbohydrates;
a source of protein;
a source of minerals to provide high alkaline ash;
the proteins selected from the group consisting of whole protein, protein concentrates and isolates which may or may not be low-acid ash protein, selected from the group consisting of pea, caseinoglycomacropeptide, carob, soya, canola, flax, wheat, corn, and potato protein and comprises pea protein in an amount of at least 20% by weight protein; and
the formulation is designed to provide at least 90% of a patient's daily calories.

2. The nutritional supplement of claim 1 comprising free carnitine.

3. The nutritional supplement of claim 1, wherein the formulation is designed to provide 100% of a patient's daily calories.

4. The nutritional supplement of claim 1, wherein the formulation is designed to be a complete nutritional.

5. The nutritional supplement of claim 1, wherein the formulation is designed to be an oral nutritional supplement.

6. The nutritional supplement of claim 1, wherein the formulation is designed to be a tube feed.

7. The nutritional supplement of claim 1, wherein the formulation is a module that can be added to any tube feed to increase the alkalinity of a consumer's diet.

8. The nutritional supplement of claim 1 comprising at least one ingredient selected from the group consisting of a prebiotic, soluble fiber, insoluble fiber, probiotic, amino acid, fish oil, phytonutrient, antioxidant, and combinations thereof.

9. The nutritional supplement of claim 8, wherein the amino acid is selected from the group consisting of Lysine, Arginine, Histidine, Glutamine, Glycine, and combinations thereof.

10. A method of reducing metabolic acidosis, complications resulting from acidosis, or conditions that may be improved by modulating the acid-base balance of a mammal comprising: administering to a mammal in need of same a nutritional supplement formulation comprising: a source of fats; a source of carbohydrates; a source of protein; a source of minerals to provide high alkaline ash; the proteins selected from the group consisting of whole protein, protein concentrates and isolates which may or may not be low-acid ash protein, selected from the group consisting of pea, caseinoglycomacropeptide, carob, soya, canola, flax, wheat, corn, and potato protein and comprises pea protein in an amount of at least 20% by weight protein; and the formulation is designed to provide at least 90% of a patient's daily calories.

11. The method of claim 10 wherein the mammal is a patient undergoing a long-term tube feeding regimen.

12. The method of claim 10 wherein the mammal is a patient having a renal insufficiency.

13. The method of claim 10 wherein the mammal is a patient at risk of a renal insufficiency.

14. The method of claim 10 wherein the mammal is a patient at risk of musculoskeletal decline.

15. The method of claim 10 wherein the mammal is a patient undergoing a parenteral nutrition regimen in combination with an enteral nutrition regimen wherein each regimen is the nutritional supplement.

16. The method of claim 10 wherein the mammal is a patient having acidosis and the nutritional supplement buffers the acidosis.

17. A method of selecting a nutritional composition for administration to a patient who can benefit from same, the method comprising:
providing a protein selected from the group consisting of whey, chicken, corn, caseinate, wheat, flax, soy, carob, pea and combinations thereof;
calculating an acid content of the nutritional composition using the equation: acid content=[(P×0.0366)+(protein (g/day) x acid potential of the protein (mEq/100g protein))+(Cl×0.0268)];
calculating a base content of the nutritional composition using the equation: base content=[(Ca×0.0125)+(Mg×0.0263)+(K×0.0211)+(Na×0.0413)];
subtracting the base content from the acid content to obtain a potential renal acid load (PRAL) value; and
selecting the nutritional composition for administration to the patient if the PRAL value is negative, wherein
P=Phosphorous content of the nutritional composition (mg/day) (for added alkalinity in contrast to original formula)
Acid potential=2×[(mg methionine present in 100 g of the protein/149.2 (g/mol))+(2×(mg cystine present in 10 0g of the protein/240.3 (g/mol)))],
Cl=Chloride content of the nutritional composition (mg/day),
Ca=Calcium content of the nutritional composition (mg/day),
Mg=Magnesium content of the nutritional composition (mg/day),
K=Potassium content of the nutritional composition (mg/day), and
Na=Sodium content of the nutritional composition (mg/day).

18. The method of claim 17, wherein the nutritional composition is in an administrable form selected from the group consisting of pharmaceutical formulations, nutritional formulations, tube-feed formulations, dietary supplements, functional foods and beverage products.

19. The method of claim 17, wherein the nutritional composition is a complete nutritional.

20. The method of claim 17, wherein the administration is a long-term administration.

21. The method of claim 17, wherein the patient has or is at risk of having a renal insufficiency.

22. The method of claim 17, wherein the patient has acidosis.

23. The method of claim 22 wherein the nutritional composition buffers the acidosis.

24. The method of claim 17, wherein the formulation is treats and/or prevents bone loss in a patient.

25. A computer implemented process for determining a potential renal acid load (PRAL) value, the process comprising:
providing a computer having an input device and a computer processor so constructed and arranged to:
a) calculate an acid content of a nutritional composition using the equation: acid content=[(P×0.0366)+(protein (g/day) x acid potential of the protein (mEq/100 g protein))+(Cl×0.0268)],
b) calculate a base content of the nutritional composition using the equation: base content=[(Ca×0.0125)+(Mg×0.0263)+(K×0.0211)+(Na×0.0413)], and
c) subtract the base content from the acid content to obtain the PRAL value, wherein the protein is selected from the group consisting of whey, chicken, corn, caseinate, wheat, flax, soy, carob, pea and combinations thereof, and wherein
P =Phosphorous content of the nutritional composition (mg/day),
Acid potential=2×[(mg methionine present in 100 g of the protein/149.2 (g/mol))+(2×(mg cystine present in 100 g of the protein/240.3 (g/mol)))],
Cl=Chloride content of the nutritional composition (mg/day),
Ca=Calcium content of the nutritional composition (mg/day),
Mg=Magnesium content of the nutritional composition (mg/day),
K=Potassium content of the nutritional composition (mg/day), and
Na=Sodium content of the nutritional composition (mg/day).

26. The process of claim 25 further including using the input device to input values for each of the phosphorous, chloride, calcium, magnesium, potassium and sodium contents of the nutritional composition.

27. The process of claim 25 further including using the input device to input an acid potential of a protein selected from the group consisting of whey, chicken, corn, caseinate, wheat, flax, soy, carob, pea and combinations thereof.
